(19) United States
(12) Patent Application Publication
(10) Pub. No.: US 2008/0014170 A1
(43) Pub. Date: Jan. 17, 2008

(54) Drug delivery polyanhydride composition and method

(76) Inventors: Olexander Hnojewyj, Emerald Hills, CA (US); Patrick Rivelli, Palo Alto, CA (US); Tony B. Shaffer, Alameda, CA (US)

Correspondence Address: PERKINS COIE LLP, P.O. BOX 2168, MENLO PARK, CA, 94026, US

(21) Appl. No.: 11/486,501

(22) Filed: Jul. 14, 2006

(51) Int. Cl. A61K 31/765; C08L 67/04

(52) U.S. Cl. 424/78.37; 525/437; 525/438

(57) ABSTRACT

The disclosure is generally directed to a biodegradable polymer composition comprising a drug-containing polymer component having a backbone containing one or more therapeutic drugs that are linked together by anhydride linkages, such that molecules of the drug are released upon biodegradation of the component. The biodegradable polymer composition also comprises a polyester-based polymer component.

### FIELD OF THE INVENTION

The present invention is generally directed to a biodegradable polymer composition comprising a drug-containing polymer component having a backbone containing one or more therapeutic drugs that are linked together by anhydride linkages, such that molecules of the drug are released upon biodegradation of the drug-containing component.

### BACKGROUND OF THE INVENTION

Biodegradable polymers are being used for many applications in medicine, including as a carrier for controlled-release drug delivery systems, and in biodegradable bone pins, screws, and scaffolds for cells in tissue engineering. A principal advantage of the materials based on biodegradable polymers over existing non-biodegradable polymers or metal-based material is that the products are removed over time by bioerosion, avoiding the need for surgical removal.

Despite the growing need in medical applications, only few synthetic biodegradable polymers are currently used routinely in humans as carriers for drug delivery: ester copolymers of lactide, lactone and glycolide (PLA family) and anhydride copolymers of sebacic acid (SA) and 1,3-bis-(carboxyphenoxy)propane (CPP). PLA is the most widely used due to its history of safe use as surgical sutures and in current drug delivery products like the Lupron Depot 19. While the development of PLA remains among the most important advances in medical biomaterials, there are some limitations that significantly curtail its use, and in particular:

(1) PLA polymers typically take a few weeks to several months to completely degrade in the body, but the device is typically depleted of drug more rapidly;

(2) PLA devices undergo bulk erosion, which leads to a variety of undesirable outcomes, including exposure of unreleased drug to a highly acidic environment;

(3) it is difficult to release drugs in a continuous manner from PLA particles owing to the polymers' bulk-erosion mechanism; and,

(4) the particularly fine PLA particles needed for intravenous injection or inhalation can agglomerate significantly, making resuspension for injection or aerosolization for inhalation difficult.

Polyanhydrides, because of their more labile polymer bond, show a more rapid degradation rate and also tend to exhibit surface, rather than bulk degradation. Because of these advantages, polyanhydrides polymers may be preferred in biological applications where it is critical to achieve a high degradation rate and/or a better controlled rate of erosion from the polymer surface.

More recently, mixed polyester/polyanhydride polymers that combine the release characteristics of both polyester and polyanhydride polymers have been proposed. See, for example, Storey, R. et al., J. Macromol Sci., Pure Appl. Chem., A34(2) pp 265-280 (1997), U.S. Pat. No. 5,756,652, and Korhonen, H. et aL, Macromol Chem. Phys., 205, pp 937-945 (2004). These polymers may be thought of as containing a selected proportion of ester and anhydride linkages along the polymer backbone chains. Increasing the proportion of anhydride linkages in the mixed polymers leads to enhanced rate of surface erosion. In certain types of mixed polyester/polyanhydride polymers, at least, the rate of erosion was found to be biphasic, evidencing a relatively rapid release of polyester components and a slower breakdown of the released polyester moieties.

One limitation of polyanhydride polymers, however, is their relatively high stiffness, or Young's modulus of elasticity, typically in the range of 3-5 GPa, making these polymers unsuitable for applications in which polymer expansion or bending is required. One important area where an expandable polymer would be useful is intravascular stents, which are carried on balloon catheters and deployed at a site of vascular injury by radial expansion, requiring the ability to expand significantly, and once expanded to hold their shape within a vessel. These physical requirements have limited stent construction heretofore largely to metal-lattice construction.

It would thus be desirable to provide a biocompatible, biodegradable polymer having improved biodegradation and drug-release properties. It would also be desirable to provide a biocompatible, biodegradable stent having the requisite deformability and shape-retention, but also capable of biodegrading over a desired “stenting” period and exhibiting surface rather than bulk erosion.

### SUMMARY OF THE INVENTION

The invention is generally directed to a biodegradable polymer composition comprising a drug-containing polymer component having a backbone containing one or more therapeutic drugs that are linked together by anhydride linkages, such that molecules of the drug are released upon biodegradation of the component. The biodegradable polymer composition also comprises a polyester-based polymer component having a molecular weight ranging from about 2 to about 20 KDaltons. The composition can be characterized by at least one of (i) a Young's modulus of no greater than about 3 GPa, and (ii) a weight ratio of the polyester-based polymer component between about 80% to about 98%.

In some embodiments, the composition comprises a blend of the drug-containing polymer component and the polyester-based polymer component. In some embodiments, the polyester-based polymer component comprises a component selected from a group consisting of a poly(lactide), a poly(glycolide), or a poly(caprolactone). In some embodiments, the number of anhydride linkages in the drug-containing polymer component is between 5 and 25. In some embodiments, the therapeutic drug in the drug-based polymer component is selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates.

In some embodiments, the composition is formed by linking the drug-containing polymer component and the polyester-based polymer component to one another through anhydride linkages. In some embodiments, the composition includes a linker unit linking the drug-containing and polyester-based polymer components through anhydride linkages. The linker unit can be selected from the group consisting of an oligomer of an ether, an amide, an ester, an anhydride, a urethane, a carbamate, a carbonate, a hydroxyalkanoate, or an azo compound. The linker unit can also contain in its polymer backbone a second therapeutic drug linked in the backbone by biodegradable linkages.

In some embodiments, the biodegradable polymer composition is characterized by a Young's modulus ranging from about 1.5 to about 3 GPa; and a rate of surface degradation that is effective to fully erode a bar of the polymer having dimensions of about 50 microns×50 microns×2 mm, when incubated in phosphate buffered saline at 37° C., within a period ranging from about 5 to about 365 days.

In some embodiments, the polyester-based polymer component has the form,

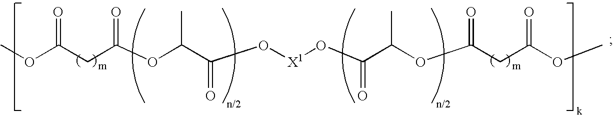

and, the drug-containing polymer component has the form,

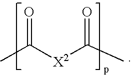

In these embodiments, X¹ comprises a component selected from a group consisting of a substituted, unsubstituted, hetero-, straight-chained, branched, cyclic, saturated or unsaturated aliphatic radical; a substituted, unsubstituted, or hetero-aromatic radical; a releasable agent; or a combination thereof; such that X¹ has a molecular weight of less than about 2,000 Daltons. In these embodiments, X² comprises a therapeutic agent and has a molecular weight of less than about 10 KDaltons, wherein the component of X¹ is the same as, or different than, the releasable agent of X². The k and p are integers selected such that the composition comprised of about 80% to about 98% by weight of the polyester-based polymer component; m is an integer ranging from 1 to 10; and n is an integer selected such that the molecular weight of each polyester-based polymer component ranges from about 2 to about 20 KDaltons.

In some embodiments, the polyester-based polymer component has the form,

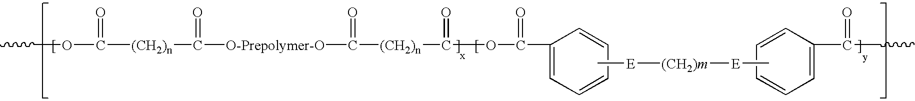

wherein,

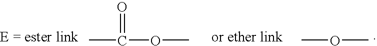

The E is a para ester or a meta or para ether linkage, and the pre-polymer is an α-ω,-dihydroxy terminated polyester or polyether polymer having a molecular weight in a selected range between 1 to 10 Kdaltons. The x is 80% to 98% of the polymer by weight, the y is 20% to 2% of the polymer by weight, the n ranges from 2 to 4, the m ranges from 2 to 10; and the average total number of anhydride linkages is a selected number ranging from about 5 to about 30.

In some embodiments, the biodegradable polymer composition comprises a drug-containing polymer component having a backbone containing one or more therapeutic drugs that are linked together by biodegradable linkages, such that molecules of the drug are released upon biodegradation of the composition. In these embodiments, the drug-containing polymer component has the form,

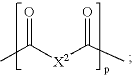

and a polyester-based polymer component having an average molecular weight of between 2 and about 20 KDaltons and having the form,

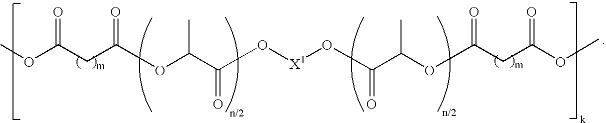

In these embodiments, polymer components are linked together through anhydride linkages to form a polyanhydride composition. The X¹ comprises a component selected from a group consisting of a substituted, unsubstituted, hetero-, straight-chained, branched, cyclic, saturated or unsaturated aliphatic radical; a substituted, unsubstituted, or hetero-aromatic radical; a releasable agent; or a combination thereof; such that X¹ has a molecular weight of less than about 2,000 Daltons. The X² comprises a therapeutic agent and has a molecular weight of less than about 10 KDaltons, wherein the component of X¹ is the same as, or different than, the releasable agent of X². The k and p are integers selected such that the poly(anhydride) is comprised of about 80% to about 98% by weight of the polyester-based polymer component; m is an integer ranging from 1 to about 10; and, n is an integer selected such that the molecular weight of each polyester-based polymer component ranges from about 2 to about 20 KDaltons. In these embodiments, the biodegradable polymer composition can be formed by linking polymer components together through anhydride linkages to form a polyanhydride composition. The composition can also include a blend of the drug-containing polymer component and the polyester-based polymer component. The biodegradable polymer composition can further include a linker unit linking the drug-containing and polyester-based polymer components through anhydride linkages.

In some embodiments, the invention is a method of producing a biodegradable polymer composition containing a therapeutic drug in bio-releasable form and having a selected Young's modulus of no greater than about 3.5 GPa. In these embodiments, the method comprises mixing a drug-containing polymer component having a backbone containing terminal anhydride groups and one or more therapeutic drugs that are linked together by biodegradable linkages, such that molecules of the drug are released upon biodegradation of the composition, with a polyester-based polymer component having a molecular weight ranging from about 2 to about 10 KDaltons and terminal anhydride groups. The weight percentage of the polyester-based polymer component ranges from about 80% to about 98% of the mixture. In these embodiments, the method can further include polymerizing the polymer components under time and temperature conditions effective to produce a polyester-based polyanhydride having a selected average number of anhydride linkages in the range of about 5 to about 25. In these embodiments, the polyester-based polymer components can have an average molecular weight ranging from about 5 to about 7 KDaltons. The polyester-based polymer component can comprise a component selected from a group consisting of a poly(lactide), a poly(glycolide), or a poly(caprolactone), and the composition can have a Young's modulus ranging from about 1.5 to about 3 GPa and a rate of surface degradation that is effective to fully erode a bar of the polymer having dimensions of about 50 microns×50 microns×2 mm, when incubated in phosphate buffered saline at 37° C., within a period ranging from about 5 to about 365 days.

In some embodiments, the invention is an expandable intravascular stent comprising an expandable stent body and an outer, drug-eluting coating. The coating is composed of a drug-containing polymer component having a backbone containing one or more therapeutic drugs that are linked together by biodegradable linkages, such that molecules of the drug are released upon biodegradation of the composition; a polyester-based polymer component having a molecular weight ranging from about 2 to about 20 KDaltons; and an anti-restenosis compound. In these embodiments, the coating is characterized by at least one of (i) a Young's modulus of no greater than about 3 GPa, and (ii) a weight ratio of the polyester-based polymer component ranging from about 80% to about 98% of the total polymer weight of the composition. In these embodiments, the stent's expandable body can be formed of a poly(ester)-based polymer component linked by anhydride linkages, and can have a Young's modulus of less than about 3 GPa.

In some embodiments, the invention is a stent comprising an expandable body; a first layer on a surface of the stent comprising rapamycin, or a derivative thereof; and a second layer on at least a portion of the first layer comprising an NSAID. In these embodiments, at least one of the expandable body, the first layer, or the second layer comprises a biodegradable polymer composition comprising a drug-containing polymer component having a backbone containing one or more therapeutic drugs that are linked together by biodegradable linkages, such that molecules of the drug are released upon biodegradation of the composition; and a polyester-based polymer component having a molecular weight ranging from about 2 to about 20 KDaltons. In these embodiments, the composition is characterized by at least one of (i) a Young's modulus of no greater than about 3 GPa, (ii) a rate of surface degradation that is effective to fully erode a bar of the polymer having dimensions of about 50 microns×50 microns×2 mm, when incubated in phosphate buffered saline at 37° C., within a period of about 5 to about 365 days, or (iii) a weight ratio of the polyester-based polymer component that is between about 80% to about 98%.

These and other objects and features of the present invention will become more fully apparent when the following detailed description is read in conjunction with the accompanying drawings.

### BRIEF DESCRIPTION OF THE DRAWINGS

FIG. 1 illustrates steps in the synthesis of an α-ω,-dihydroxy polylactide prepolymer having a diethylene glycol core, and the conversion of the dihydroxy prepolymer to a dicarboxylic acid prepolymer;

FIG. 2 illustrates steps in the conversion of the dicarboxylic acid prepolymer of FIG. 1 to an α-ω,-dianhydride polylactide prepolymer, and its polymerization to yield a polyanhydride polymer;

FIG. 3 illustrates steps in the synthesis of a polyethyleneglycol-based polyanhydride;

FIG. 4 illustrates steps in the synthesis of a 1,3-bis(p-carboxyphenoxy)propane subunit;

FIG. 5 illustrates steps in the synthesis of a polyanhydride copolymer of polylactide and the 1,3-bis(p-carboxyphenoxy)propane subunit of FIG. 4; and

FIGS. 6A and 6B illustrate in perspective (6A) and cross-section (6B) a stent constructed in accordance with the invention;

FIG. 7 is a plot showing the rates of degradation of (i) a PLA polymer, (ii) a PLA polyanhydride polymer constructed in accordance with the invention; and (iii) a conventional polyanhydride polymer.

### DETAILED DESCRIPTION OF THE INVENTION

I. Definitions

Unless indicated otherwise, the terms below have the following definitions:

A “polyanhydride polymer” is a polymer having at least some anhydride linkages between subunits of the polymer chain. More particularly, a polyanhydride polymer as defined herein, includes polyester or polyether subunits or blocks joined by anhydride linkages, and this polymer is also identified herein as a mixed polyester/polyanhydride or polyether/polyanhydride polymer. This polyanhydride polymer may also contain other polymer subunits or blocks, forming block copolymers whose blocks are linked by anhydride linkages. The composition of such polyanhydride co-polymers may be expressed in terms of relative weight percent of the two polymer blocks making up the block co-polymer.

A “prepolymer” refers to a polyester or polyether polymer chain which, when converted to a suitable di-functional polymer, e.g. a dicarboxylic acid, forms a polymer subunit, which can be a block or repeating unit, for example, in a polyanhydride polymer. A polymer subunit can have its own repeating units, such as a polyester or polyether repeating unit, and can also have a core, such as a poly(ethylene glycol) core that can be used to join other chemical moieties, such as polyester moieties, for example.

The “average number of anhydride linkages” in an anhydride polymer is the average total number of anhydride linkages present connecting one or more polymer subunits in the polyanhydride chains, and may be determined, for example, by determining the average molecular weight of the anhydride polymer, knowing the relative amounts and sizes of the individual polymer blocks making up the polyanhydride polymer.

The “average molecular weight of polymer chains” in a polymer composition is the average molecular weight of the chains determined with respect to polylactide standard (from Polymer Source, Inc.) by size exclusion chromatography, according to standards methods (Ref: A. Kowalski, et. al., Macromolecules 1998, 31, 2114). Average molecular weight of the polylactide anhydrides also be measured by other means, including laser-desorption ionization time-of-flight mass spectrometry, as described Zhu, H. et al, Journal of the American Society for Mass Spectrometry, Volume 9, Number 4, April 1998, pp. 275-281(7). Viscosity average molecular weight of the polylactide anhydride can be determined by solution viscosity measured in chloroform at 35° C. using a size 4 Ubbelohde viscometer (obtained from Cannon Instrument Co.USA).

“Intrinsic viscosity” is defined as the viscosity of a polymer solution in an unlimited dilute concentration. It is independent on the concentration by virtue of extrapolation to zero concentration. In practice, when the polymer solution is dilute enough to separate the chains from each other by solvent, the relative viscosity (ηr) and specific viscosity (ηsp) will follow the following equations:

ηsp/c=[η]+k′[η]²c

ln ηr/c=[η]+k″[η]²c

where:

ηr=η solution/η solvent

ηsp=(η solution/η solvent)−1

Within the dilute concentration range, intrinsic viscosity can be obtained by plotting ηsp/c vs. c and ln ηr/c vs c to extrapolate the line to c=0.

The relationship between intrinsic viscosity and molecular weight can be found in Mark-Houwink equation:

[η]=κM^{α}

where κ and α are parameters related to type of polymer, solvent and temperature. The molecular weight can be calculated from intrinsic viscosity if the parameters are known. In the present case, for example, the poly(D/L-lactide) based polyanhydride can be considered as pure poly(D/L-lactide) with several anhydride linkages instead of ester linkages. The parameters of poly(D/L-lactide) can be used to estimate the molecular weight of polyanhydride.

The number of anhydride linkages in a polymer chain can be estimated from the molecular weight of the polyanhydride divided by the molecular weight of the pre polymer. The average number of anhydride linkages can also be determined from Light Scattering detectors attached on line with size exclusion chromatography.

The size of the macromolecule is large enough to cause light scattering, which can be used to calculate the molecular weight. Combining size exclusion chromatography (SEC) and light scattering on-line detector gives a rapid, efficient way to determine molecular weight and molecular weight distribution. Unlike pure polylactide, a polylactide anhydride can have difficulty eluting through a column packing material. This might be due to strong adsorption of the polyanhydride chains with the packing material.

In the determination of the molecular weights of the polyanhydride, the SEC columns are disconnected and a known concentration of polyanhydride is directly injected to the Viscotek T60A dual detector (Visco-LS) and the Varian 9040 RI detector with a guard column between the sample injector and the detectors. Chloroform (dried on CaH₂) or THF (dried over a benzophenone/Na complex) is used as the eluent at a flow rate of 1 ml/min. The dη/dc of the polymer was calculated in CHCl₃ and in THF. The molecular weight, intrinsic viscosity and radius of gyration were then analyzed by the Viscotek TriSEC software.

“Young's modulus” or “Young's modulus of elasticty” is a measure of the stiffness of a given material. This can be experimentally determined from the slope of a stress-strain curve created during tensile tests conducted on a sample of the material, and is usually expressed in GPa, i.e., 10¹² N/m². Relatively stiff polymers, such as conventional polyanhydrides, polystyrene, and polyimides, have Young's modulus values in the range 3-5. Soft or highly flexible polymers, such as polyethylene, or rubber, can have Young's modulus values below 1. Young's modulus measurements can be made, for example, as described in L. A. Carlsson et al., Experimental Characterization of Advanced Composite Materials, Chapter 3 and 4, and ASTM Standard #E111-4 method, as detailed, for example, in the ACTIVE STANDARD: E111-04 Standard Test Method for Young's Modulus, Tangent Modulus, and Chord Modulus, available form ASTM international (http://www.astm.org/cgi-bin/SoftCart.exe/DATABASE.CART/REDLINE_PAGES/E111.htm?E+mystore)

### II. Synthesis of polyester/polyanhydride polymers

The biocompatible, biodegradable polyanhydride polymers of the invention may be useful in applications, such as, for example, the delivery of biologically active compounds, preparing films, coatings, medical implants, coatings for medical implants and the like. The polymers can be readily processed into pastes or films, coatings, microspheres and fibers with different geometric shapes. The polymers can be processed into finished articles or coatings using techniques known in the art, such as, for example, solvent casting, spraying solutions or suspensions, compression molding and extrusion. Medical implant applications include the use of the polyanhydrides to form shaped articles, such as vascular grafts, stents, bone plates, sutures, implantable sensors, and other articles that decompose into non-toxic components over a known time period. In addition, the polymers can be used to form coatings for those articles, which may require the release of an active compound.

Polymers prepared from the process of the present invention can also be incorporated into oral formulations and into products such as skin moisturizers, cleansers, pads, plasters, lotions, creams, gels, ointments, solutions, shampoos, tanning products and lipsticks for topical application. As described herein, the polymer compositions of the invention generally include a polyester-based polymer component and a drug-containing polymer component.

A. Preparing the Polyester-Based Polymer Component

In many embodiments, the synthesis of the polymers of the invention can proceed in three steps. First, one or more prepolymer blocks or block subunits are provided, e.g., by synthesis. As will be seen further below, one of the prepolymers, and typically the dominant polymer subunit in the polyanhydride polymer, is preferably a polyester prepolymer having a molecular weight in the range of 1-10 Kdaltons. In embodiments where the polyanhydride polymer has a desired Young's modular of elasticity in the range of 1.5-3, the prepolymer can have a molecular weight that is greater than 5 Kdaltons, less than about 10 Kdaltons, and can also range from about 6-7 Kdaltons. The polyester-based polymer component can be a monomer, oligomer, or polymer.

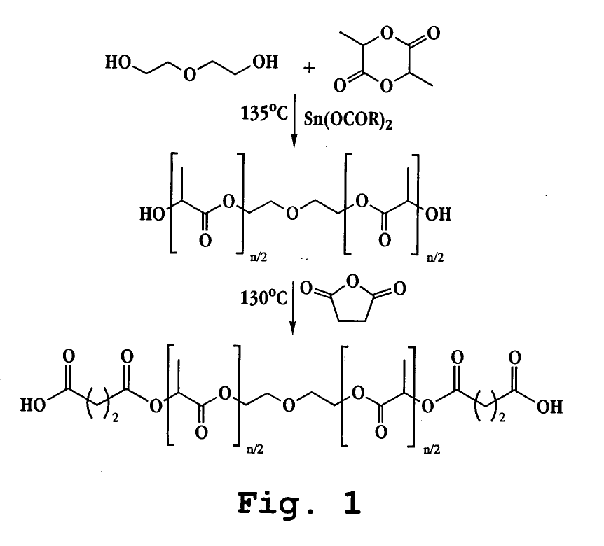

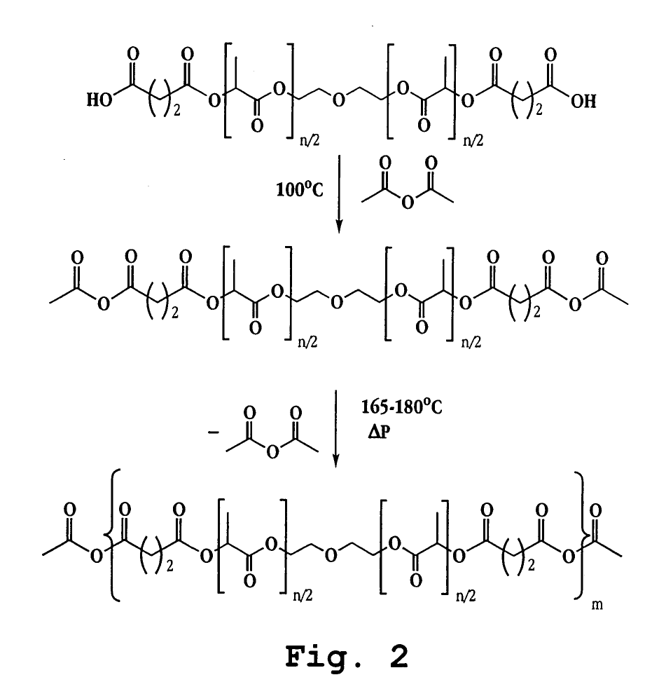

The polyester prepolymer may include a non-polyester core, for example, a dihydric alcohol core, such as a diethylene glycol core, as seen below in Examples 1 and 2 and with respect to FIGS. 1 and 2. Preferred polyester polymers can include biocompatible, bioerodable polyester polymers, such as poly(lactic acid) (PLA, or polylactide), polyglycolic acid (PGA), and poly(ε-caprolactone), each often containing a dihydric alcohol core. Methods for synthesizing such polyester or polyether or mixed polyether/polyester polymers are well-known in the art, and one exemplary method is described in Example 1 below with reference to FIG. 1.

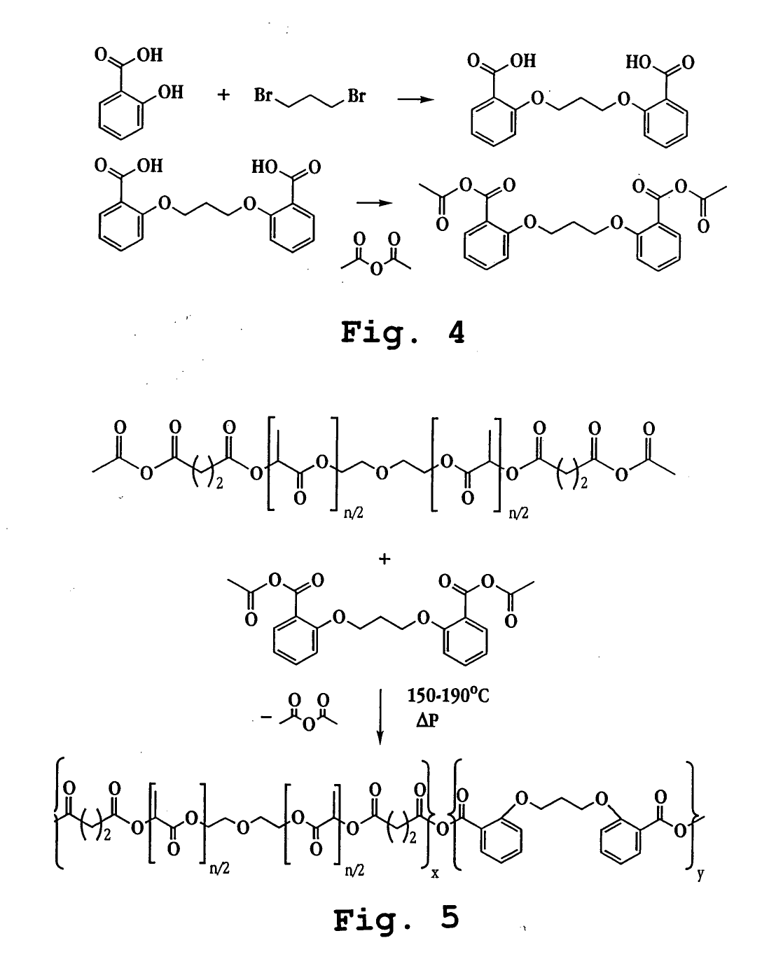

In addition to a polyester (and/or polyether) prepolymer component(s), the polyanhydride polymer of the invention may contain other block components including, but not limited to, diphenoxy subunits, such as the 1,3-bis(carboxyphenoxy)propane subunit whose synthesis is described in Example 4 with respect to FIG. 4. In an anhydride polymer whose subunits include both a polyester and at least one other block, e.g., a diphenoxy subunit, the polyester is preferably present in an amount ranging from about 80%−98 percent by weight of the final polymer, with the other component(s) being present in an amount ranging from about 2-20% by weight of the final polymer.

In the second step in forming the polyanhydride of the invention, the prepolymer component(s) from above are converted to terminal-group dicarboxylic acids, e.g., from α-ω,-dihydroxy terminated polyester prepolymers, to corresponding α-ω,-dicarboxylic acid terminated prepolymers. This conversion is typically carried out by reaction of the prepolymer with succinic anhydride. More generally, a reaction of α-ω,-dihydroxy terminated polyester (or polyether) polymers with the cyclic anhydride, for example, produces α-ω,-dicarboxylic acid terminated polyester (or polyether) prepolymers, according to known methods. Methods for converting polyester or polyether or mixed polyether/polyester polymers to corresponding dicarboxylic acids are well-known in the art. Exemplary methods are described below in Example 2 with respect to reference to FIG. 2.

In the final polymerization step, the prepolymer components of the polymer are polymerized under conditions effective to link those components by anhydride linkages. This is done, in one exemplary method, by first reacting the dicarboxylic acid prepolymer or block components with acetic anhydride, to convert the terminal acid groups to corresponding anhydrides. The prepolymer dianhydrides are then dried to remove unreacted acetic anhydride. In the final polymerization step, the dianhydride block or prepolymer components are mixed in a desired weight proportion, as noted above, and reacted under conditions effective to produce a polyanhydride polymer having a selected number of polyanhydride linkages, e.g., 3-30 anhydride linkages.

One exemplary polymerization method is the method described below in Example 2 with reference to FIG. 2. Briefly, in this method, the dianhydride component(s) are added to a metal oxide, such as calcium oxide, and heated under an inert atmosphere until melting, with continued heating under vacuum to remove excess acetic anhydride, with additional heating, e.g., at a temperature between 180° C. to 220° C., until a desired degree of polymerization has occurred.

The degree of polymerization, that is, the number of anhydride linkages in the final polymer can be determined readily from intrinsic viscosity of the polymer and by light scattering measurement from Viscotek detectors, as described above, to determine polyanhydride molecular weight, then dividing by the known molecular weight of the pre polymer. As will be seen below, the desired extent of polymerization will be dictated by elasticity properties and rates of degradation that are desired. For example, in accordance with one embodiment of the invention, it has been discovered that a high polyanhydride elasticity (a low Young's modulus) can be achieved in a polyester prepolymer having an average molecular weight of about 6 Kdaltons, and between 8-12 anhydride linkages. A polymer having more than 8-12 linkages will show a greater rate of surface degradation, and also a greater Young's modulus. Thus, in accordance with this embodiment of the invention, biocompatible, biodegradable polymers having a desired elasticity and surface degradation rate can be achieved by certain reaction variables that are readily selected, including:

(1) the molecular weight of the polyester (or polyether) prepolymer which, as noted above, and seen from the data in Section III below, a high polyanhydride elasticity (a low Young's modulus) can be achieved at a polyester prepolymer molecular weight of greater than 5 Kdaltons and less than about 7-10K daltons;

(2) the extent of polymerization, as measured by the average number of anhydride linkages in the final polymer, which will effect both elasticity and rate of surface degradation; and

(3) the presence of block components other polyester prepolymers such as, for example, where the polyanhydride includes the 1,3,-bis(carboxyphenoxy)propane component described in Example 4, can have the effect of improving the bioerosion characteristics of the polymer to favor bioerosion over bulk erosion, for example.

B. Preparing the Drug-Containing Polymer Component

Drugs can be blended and/or otherwise combined with the polymers of the invention including, but not limited to, incorporating the drugs into the backbone of the polymer. Incorporating drugs into the backbone can include a process of creating a drug-containing polymer component and polymerizing the drug-containing polymer component with a polyester-based polymer component to form a polymer of the invention.

The drug-containing polymer component can provide a therapeutic benefit upon biodegradation from a polymer composition into the body. The rate of release of a drug can be controlled by the design of the polymeric matrix, such that the degradation, or lack thereof, and the resulting morphology of the polymeric matrix can dictate diffusion of the drug from the polymeric matrix into a body. The type of bond, or lack of bond, that is used to combine the drug with the polymeric matrix also affects release rate. An anhydride bond is more labile than an ester bond, an ester bond is more labile than an amide bond, and an amide bond is more labile than an ether bond, for example. Electron donating and withdrawing groups can also be implemented for additional control. Chemical additive groups that affect the hydrophilicity of the polymeric matrix can also be added to control diffusion by assisting in the introduction of water as a diffusion medium throughout the polymeric matrix. An example of such a group is poly(ethylene glycol) (PEG), which can be added as part of the polymer as a pendant group or in-chain group. Poly(ethylene glycol) can sometimes be effective when blended with the polymer matrix.

Drugs that can be used in some embodiments of the invention include, but are not limited to, antiproliferatives, antineoplastics, antimitotics, anti-inflammatories, antiplatelets, anticoagulants, antifibrins, antithrombins, antibiotics, antiallergics, antioxidants, analgesics, anesthetics, antipyretics, antiseptics, and antimicrobials. It is to be appreciated that one skilled in the art should recognize that some of the groups, subgroups, and individual bioactive agents may not be used in some embodiments of the present invention.

Antiproliferatives include, for example, actinomycin D, actinomycin IV, actinomycin I₁, actinomycin X₁, actinomycin C₁, and dactinomycin (COSMEGEN®, Merck & Co., Inc.). Antineoplastics or antimitotics include, for example, paclitaxel (TAXOL®, Bristol-Myers Squibb Co.), docetaxel (TAXOTERE®, Aventis S.A.), methotrexate, azathioprine, vincristine, vinblastine, fluorouracil, doxorubicin hydrochloride (ADRIAMYCIN®, Pfizer, Inc.) and mitomycin (MUTAMYCIN®, Bristol-Myers Squibb Co.), and any prodrugs, metabolites, analogs, homologues, congeners, derivatives, salts and combinations thereof. Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, derivatives, salts and combinations thereof.

Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, metabolites, analogs, homologues, congeners, derivatives, salts and combinations thereof. Antiallergic agents include, but are not limited to, pemirolast potassium (ALAMAST®, Santen, Inc.), and any prodrugs, metabolites, analogs, homologues, congeners, derivatives, salts and combinations thereof.

Other bioactive agents useful in the present invention include, but are not limited to, free radical scavengers; nitric oxide donors; rapamycin; everolimus; tacrolimus; 40-O-(2-hydroxy)ethyl-rapamycin; 40-O-(3-hydroxy)propyl-rapamycin; 40-O-[2-(2-hydroxy)ethoxy]ethyl-rapamycin; tetrazole containing rapamycin analogs such as those described in U.S. Pat. No. 6,329,386; estradiol; clobetasol; idoxifen; tazarotene; alpha-interferon; host cells such as epithelial cells; genetically engineered epithelial cells; dexamethasone; and, any prodrugs, metabolites, analogs, homologues, congeners, derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (TEMPOL), 2,2′,3,4,5,5′-hexamethyl-3-imidazolinium-1-yloxy methyl sulfate, free radical; 16-doxyl-stearic acid, free radical; superoxide dismutase mimic (SODm) and any analogs, homologues, congeners, derivatives, salts and combinations thereof. Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, derivatives, salts and combinations thereof.

In some embodiments, the drugs that can be used to form a drug-containing polymer component include, but are not limited to, salicylic acid, 4-aminosalicylic acid, 5-aminosalicylic acid, 4-(acetylamino)salicylic acid, 5-(acetylamino)salicylic acid, 5-chlorosalicylic acid, salicylsalicylic acid (salsalate), 4-thiosalicylic acid, 5-thiosalicylic acid, 5-(2,4-difluorophenyl)salicylic acid (diflunisal), 4-trifluoromethylsalicylic acid, sulfasalazine, dichlofenac, penicillamine, balsalazide, olsalazine, mefenamic acid, carbidopa, levodopa, etodolac, cefaclor, and captopril.

The drug-containing polymer component can be a monomer, oligomer, or polymer. An unprotected salicylate, for example, can be directly coupled to a diacyl halide, which acts as a linker to join salicylate units to form a dimer of the salicylate, and the dimer is a polymerizable subunit. The coupling of the salicylate monomers occurs in the presence of at least about 2 equivalents to about 50 equivalents of an organic base such as, for example, pyridine and the like in a suitable solvent, such as, for example, tetrahydrofuran (THF), dimethyl formamide (DMF) or mixtures thereof, to prepare the salicylate for polymerization into a drug-containing polymer component.

In one embodiment, the process uses solvents such as tetrahydrofuran (THF) and N,N-dimethyl formamide (DMF), in the presence of stoichiometric pyridine. In another embodiment, there is an excess of pyridine or the pyridine is used as a co-solvent, e.g., 3 parts THF to 1 part pyridine, by volume). In another embodiment, there is no solvent other than the organic base. The dimer of salicylate is a dicarboxylic acid that can be polymerized in the manner discussed herein with respect to the 1,3-bis(carboxyphenoxy)propane subunit, to form a drug-containing polymer component of the present invention. The diacyl halide is an example of a linker, and other linkers can be used, as described herein.

A multitude of low molecular weight therapeutically drugs can be used in the present invention, such as, for example, those disclosed in U.S. Pat. No. 6,486,214, which is hereby incorporated herein by reference in its entirety. Drugs, which can be linked into degradable co-polymers via the polyanhydrides, often have the following characteristics: a relatively low molecular weight of approximately 1,000 Daltons or less, at least one carboxylic acid group, and at least one hydroxy, amine, or thiol group.

A combination of drugs can be administered using the methods of the present invention. A second drug, for example, can be (i) dispersed in the polymeric matrix and released upon degradation; (ii) appended to a polymer as a sidechain, for example; and/or (iii) incorporate into the backbone of a polymer in the polyester-based polymer component, the drug-containing polymer component, or a linker.

Dosages of the drugs can be determined using techniques well-known to one of skill. For example, one of skill can use in vitro or in vivo activity of a drug in animal models. The extrapolation of effective doses in mice to humans, for example, is a technique that is well-known in the art. See, for example, U.S. Pat. No. 4,938,949, which is hereby incorporated herein by reference in its entirety. Moreover, the rates of release due to hydrolysis of a drug from the polymer should also be considered, which can vary by selection of polymer, method of administration, and route of administration, as well as by the age and condition of the patient.

C. Preparing the Polymer Compositions

The compositions of the present invention include any combination of polymers, copolymers and agents, wherein the combination comprises a polyanhydride polymer as taught herein. These polymers are biodegradable due to the labile nature of chemical moieties that are present. Accordingly, these compositions can be designed such that they can be broken down, absorbed, resorbed and eliminated by a mammal including, but not limited to, a human. The compositions of the present invention can be used, for example, to form pharmaceutical dosage forms, medical devices, or coatings.

For the purposes of the present invention, a polymer or coating is “biodegradable” when it is capable of being completely or substantially degraded or eroded when exposed to either an in vivo environment or an in vitro environment having physical, chemical, or biological characteristics substantially similar to those of the in vivo environment within a mammal. A polymer or coating is capable of being degraded or eroded when it can be gradually broken-down, resorbed, absorbed and/or eliminated by, for example, hydrolysis, enzymolysis, metabolic processes, bulk or surface erosion, and the like within a mammal. It should be appreciated that traces or residue of polymer may remain on a device following biodegradation. The terms “bioabsorbable” and “biodegradable” are used interchangeably in this application.

The polymer compositions of the invention can comprise blends or copolymers of the polyester-based polymer component and the drug-containing polymer component. In some embodiments, each of the polyester-based polymer component and the drug-containing polymer component can have molecular weights ranging from about 5000 to about 300,000 Daltons, from about 10,000 to about 200,000 Daltons, from about 20,000 to about 150,000 Daltons, from about 30,000 to about 125,000 Daltons, from about 50,000 to about 100,000 Daltons, or any range therein. The polyester-based polymer component and the drug-containing polymer component can each serve as independent polymers in a blend. Drugs can be incorporated into each polymer, blended with the compositions, or a combination thereof.

Copolymers can be beneficial in that they can often be designed to perform in a desired manner. For example, copolymers can offer stability in performance, since some blends and mixtures can include drugs or other components that will diffuse from a polymer composition at a rate that is faster than desired due to a burst release of a drug and/or degraded polymer components. An example of a composition that is improved through copolymerization, as discussed above, is the combination of PEG with a polymer.

In some embodiments, the biodegradable polymer compositions can include a drug-containing polymer component having a backbone containing one or more therapeutic drugs that are linked together by anhydride linkages, such that molecules of the drug are released upon biodegradation of the component. The biodegradable polymer composition also comprises a polyester-based polymer component having a molecular weight ranging from about 2 to about 20 KDaltons. The composition can be characterized by at least one of (i) a Young's modulus of no greater than about 3 GPa, and (ii) a weight ratio of the polyester-based polymer component between about 80% to about 98%.

In some embodiments, the composition comprises a blend of the drug-containing polymer component and the polyester-based polymer component. The polyester-based polymer component can include a component selected from a group consisting of a poly(lactide), a poly(glycolide), or a poly(caprolactone). In some embodiments, the number of anhydride linkages in the drug-containing polymer component is between 5 and 25. In some embodiments, the therapeutic drug in the drug-based polymer component can be selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates. In some embodiments, the composition is formed by linking the drug-containing polymer component and the polyester-based polymer component to one another through anhydride linkages.

In some embodiments, the biodegradable polymer composition is characterized by a Young's modulus ranging from about 1.5 to about 3 GPa; and a rate of surface degradation that is effective to fully erode a bar of the polymer having dimensions of about 50 microns×50 microns×2 mm, when incubated in phosphate buffered saline at 37° C., within a period ranging from about 5 to about 365 days.

In some embodiments, the polyester-based polymer component has the form,

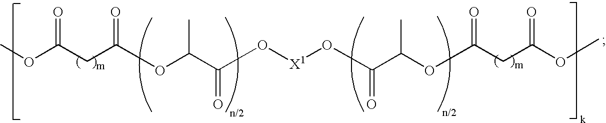

and, the drug-containing polymer component has the form,

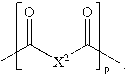

In these embodiments, X¹ comprises a component selected from a group consisting of a substituted, unsubstituted, hetero-, straight-chained, branched, cyclic, saturated or unsaturated aliphatic radical; a substituted, unsubstituted, or hetero-aromatic radical; a releasable agent; or a combination thereof; such that X¹ has a molecular weight of less than about 2,000 Daltons. In these embodiments, X² comprises a therapeutic agent and has a molecular weight of less than about 10 KDaltons, wherein the component of X¹ is the same as, or different than, the releasable agent of X². The k and p are integers selected such that the composition comprised of about 80% to about 98% by weight of the polyester-based polymer component; m is an integer ranging from 1 to 10; and n is an integer selected such that the molecular weight of each polyester-based polymer component ranges from about 2 to about 20 KDaltons.

In some embodiments, the polyester-based polymer component has the form,

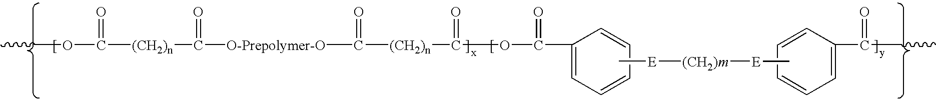

wherein,

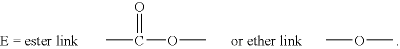

The E is a para ester or a meta or para ether linkage, and the pre-polymer is an α-ω,-dihydroxy terminated polyester or polyether polymer having a molecular weight in a selected range between 1 to 10 Kdaltons. The x is 80% to 98% of the polymer by weight, the y is 20% to 2% of the polymer by weight, the n ranges from 2 to 4, the m ranges from 2 to 10; and the average total number of anhydride linkages is a selected number ranging from about 5 to about 30.

In some embodiments, the composition includes a linker unit linking the drug-containing and polyester-based polymer components through anhydride linkages. Linkers can be used in the polyester-based polymer component, the drug-containing polymer component, or to link polymer components. The linker unit can be, for example, an oligomer of an ether, an amide, an ester, an anhydride, a urethane, a carbamate, a carbonate, a hydroxyalkanoate, or an azo compound. The linker unit can also contain in its polymer backbone a second therapeutic drug linked in the backbone by biodegradable linkages.

The linker can be chosen to biodegrade or to remain stable in the body. A biodegradable linker can have any interunit linkage such as, for example, an ester, an anhydride, an acetal, an amide, a urethane, a urea, a glycoside, a disulfide, and a siloxane linkage. It is to be appreciated that one skilled in the art should recognize that some of these linkages may not be used in some embodiments of the present invention. The selection of the linker functionality allows for control of the relative strength or stability of the bonds provided by the linker to join the polymer components. Control over this relative strength or stability can, for example, allow for some control over the release of drugs from the polymeric carrier. A stable linker can be used to maintain the integrity of at least a portion of a polymer. As the polymer biodegrades, physical properties that are provided by the polymer are affected, and a stable linker can help to maintain physical properties.

In some embodiments, polymer components can be connected by a linker, which can be a substituted, unsubstituted, hetero-, straight-chained, branched, cyclic, saturated or unsaturated aliphatic radical; and a substituted or unsubstituted aromatic radical. In some embodiments, the linker can comprise from about 0 to about 50 carbon atoms, from about 2 to about 40 carbon atoms, from about 3 to about 30 carbon atoms, from about 4 to about 20 carbon atoms, from about 5 to about 10 carbon atoms, and any range therein. In other embodiments, the linker can alternately comprise a non-carbon species such as, for example, a disulfide. In some embodiments, the linker can include substituted or unsubstituted poly(alkylene glycols), which include, but are not limited to, PEG, PEG derivatives such as PPG, poly(tetramethylene glycol), poly(ethylene oxide-co-propylene oxide), or copolymers and combinations thereof.

In some embodiments, the linker can have a molecular weight ranging from about 25 Daltons to about 1000 Daltons, from about 50 Daltons to about 750 Daltons, from about 75 Daltons to about 500 Daltons, or any range therein. In some embodiments, the linker can have a length ranging from about 5 angstroms to about 500 angstroms, from about 15 angstroms to about 400 angstroms, from about 10 angstroms to about 250 angstroms, from about 15 angstroms to about 100 angstroms, from about 25 angstroms to about 50 angstroms, or any range therein.

In some embodiments, the linker can include amino acids. In these embodiments, the amino acids can be therapeutic peptides. The therapeutic peptides can be oligopeptides, polypeptides, or proteins. Examples of amino acids that can be useful in the present invention are chemokines and chemokine analogs, such as interleukins and interferons.

### III. Applications

A. Polymer characteristics

As noted above, the present invention provides a method for producing a biodegradable, polyanhydride polymer having a selected Young's modulus between 1.5-3 GPa, and optionally, a polymer having both a selected Young's modulus and selected rate of surface degradation.

The Young's modulus of the polyanhydride polymer may be determined by standard methods, such as by the ASTM Standard #E111-4 method, as described above. Young's modulus measurements carried out on various polyanhydrides of the invention showed increasing elasticity (lower Young's modulus values) with greater polyester lengths (a polyester prepolymer with a diethyleneglycol core) with increasing prepolymer molecular weight in the molecular weight range 1-6 Kdaltons, and decreasing elasticity as the prepolymer molecular weight was increased beyond about 6-7.5 Kdaltons, at a fixed number of about 10 anhydride linkages. In general, the method of the invention will be effective in achieving Young's modulus values in the range 1.5-3, as opposed to the higher values (e.g., greater than 3 and up to 5, seen with conventional anhydride polymers.

The second variable in the polymer producing method is the number of anhydride linkages, which will affect both elasticity and rate of polymer degradation. In carrying out the method of the invention, once an optimal prepolymer length is identified, for purposes of obtaining desired elasticity properties in the polymer, the polymerization conditions can be varied to achieve a selected number of anhydride linkages, typically selected to strike a balance between achieving desired elasticity properties and surface degradation properties. The selected average number of anhydride linkages is often between 5-30, 6-25, 7-20, 8-15, and any range therein, where polyanhydride polymers having a greater number of such linkages show more rapid surface degradation rates.

To illustrate, at a polyester prepolymer molecular weight of between 6-7.5 Kdaltons, optimal flexibility is achieved under polymerization conditions that yield an average of about 8-12, and more specifically, about 10 anhydride linkages. However, if a greater surface degradation rate is desired, polymerization conditions yielding a greater number of anhydride linkages, e.g., up to 30, would be employed. As can be seen from Example 2, increasing numbers of anhydride linkages are achieved by carrying out the polymerization reaction for longer periods, e.g., up to 6-12 hours, and optionally, at somewhat higher temperatures, e.g., 170° C. preferably at 180° C.

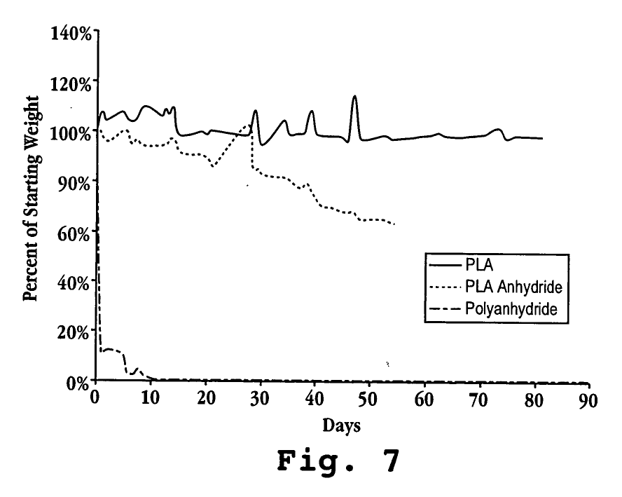

The degradation properties of the novel polyanhydrides of the invention can be seen from the degradation plots shown in FIG. 7. The graph compares the rate of degradation of a conventional PLA polymer, a conventional polyanhydride, and a polyanhydride of the invention having a prepolymer molecular weight of between 6-7.5 Kdaltons, and an average of about 8-12 anhydride linkages. Degradation rates were measured using a polymer bar having bar dimensions of 50 microns×50 microns×2 mm, incubated in phosphate-buffered saline (PBS) at 37° C. for periods of up to 100 days. At periodic test intervals, the bar was weighed to determine loss of material, and also inspected microscopically to determine whether degradation was largely occurring at the surface, as evidenced by a relatively smooth-surfaced bar, or by bulk degradation, as evidenced by the presence of pits or cavities within the bar.

As seen from FIG. 7, the PLA polymer showed little degradation after 80 days. With longer degradation times, the PLA bar showed signs of bulk degradation. The polyanhydride polymer, by contrast, was about 90% degraded after one day and completely degraded within 10 days. At all times, the bar had a smooth surface indicative of surface degradation. The polyester-based polyanhydride showed a relatively linear degradation rate that was intermediate between the other two polymers, losing about 40% of its weight after about 50 days. Extrapolation of these time points show that complete degradation would occur over a period of about 150 days. Further, inspection of the degrading polymer showed that degradation was occurring by surface, rather than bulk loss.

B. Biodegradable stents

The polyester- or polyether-based polyanhydrides of the invention have a number of biomedical applications that take advantage of the improved elasticity and/or degradation properties of the polymers. For example, the block-copolymers described with respect to FIG. 5 may be advantageous for drug-delivery because of improved bioerodability. In this application, the block co-polymers would be formed in the presence of a selected drug, at a drug/polymer ratio in the range 1:50 to 1:1, and formed into desired drug-delivery devices of particles, e.g., injectable particles having sizes in the 0.5 to 50 micron size range.

An important application of the high-flexibility polyanhydride described above is in a biocompatible, biodegradable intravascular stent. Currently stents for use at intravascular sites of injury are deployed by radial expansion over a balloon catheter, and thus require the ability to expand significantly and to hold their expanded shape when deployed, properties that led to the widespread use of metals, such as stainless steel, in stent construction. The present invention provides an expandable, shape-retaining bioerodable stent material, allowing the advantages of physical stenting, but in a device that will ultimately biodegrade by surface erosion over a selected stenting period.

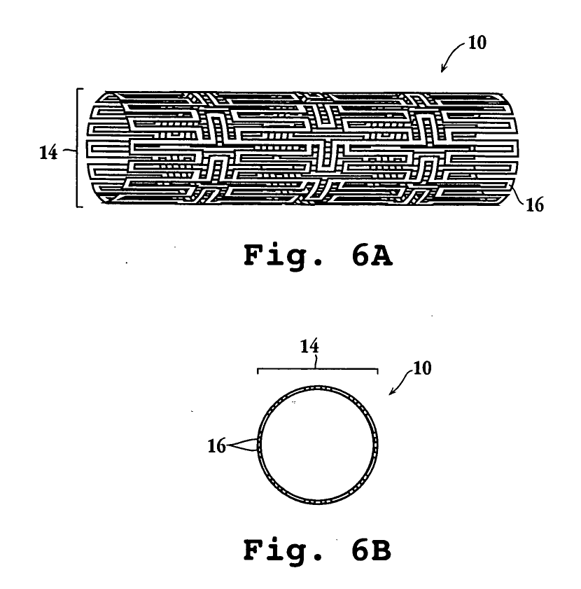

FIG. 6A shows a stent 10 constructed in accordance with a conventional stent architecture, but formed from a biocompatible, biodegradable polyanhydride material in accordance with the invention. As seen best in FIG. 6B, the stent includes a core 14 formed of a lattice of interconnected struts, such as struts 16, according to known stent architecture. This core, which is formed of an expandable, controlled-degradation polyanhydride of the invention, may be made conventionally, e.g., by forming the polymer into a cylindrical sleeve, and laser cutting the struts. Typically, the polyanhydride forming the core will have a degradation rate for complete bioerosion over a 180-360 day period.

The stent's core may be coated with a biodegradable drug-eluting coating, designed to release an anti-restensosis drug, such as taxol or rapamycin, embedded in the coating, over a selected time period. Typically, drug-elution is designed to occur over a relatively short period, e.g., 3 days to two weeks post implantation, and therefore the coating can be formed advantageously from a conventional polyanhydride with rapid surface erosion characteristics. Such a drug-containing polymer may be prepared by known methods, and applied to the stent core by conventional means, such as by dipping or spraying. The coating has a typical thickness between 3-50 microns and thus can be expanded, along with the stent core, even though the coating has a Young's modulus in the range greater than 3 GPa.

This aspect of the invention thus includes an expandable, biodegradable stent comprising a biodegradable, polyanhydride polymer having, as a repeating polymer unit, the dianhydride of a polylactide, polycaprolactone or polyglycolide α-ω,-dihydroxy polymer having an average molecular weight greater than 5 and less than 10 Kdaltons, a selected Young's modulus between 1.5 and 3, and a selected average number of anhydride linkages in the range between 5 and 25. In the embodiment just described, the polyanhydride polymer forms a biodegradable stent core which is coated, on its exterior surface(s), with a polymer coating composed of a second biodegradable polyanhydride having a Young's modulus greater than 3, and a drug embedded therein.

The following examples will illustrate various methods for synthesizing and characterizing polyanhydride polymers, in accordance with the present invention, but are in no way intended to limit the scope of the invention.

### EXAMPLE 1

### Synthesis of an α-ω,-di carboxylic acid polylactide prepolymer: Prepolymer of D/L-lactide

The steps in this example are described with reference to FIG. 1. Diethylene glycol is distilled over CaH₂ before use. D/L-lactide was sublimed under vacuum. All the solvents were purified by distillation over proper dehydrating reagent to remove the moisture. Under argon protection, 0.0238 mole of diethylene glycol, D/L-lactide (120 g) were added to a 1000 ml flask. The mixture was heated to 135° C. Once the lactide monomer was melted down, a catalyst of Tin(II) 2-ethylhexanoate (100 mg-1 ml of toluene) was added by glass syringe. The mixture was heated to 135° C. for 25 minutes. In 25 minutes, the monomer conversion reached about 90% at equilibration of polymerization with the un-reacted monomer. The reaction was stopped by cooling down the reaction flask in cold water. The solidified polymer was dissolved in acetone, and the polymer was precipitated in ethanol/hexane 2:8 v/v mixture. This procedure of precipitation was repeated three times to remove the unreacted lactide monomer.

The presence of lactide monomer in the form of a polymer was checked by FTIR by identifying the disappearance of a characteristic absorbance at 1250 cm⁻¹ from the cylic structure of the monomer. The yield of the polymer was 109 g. The SEC and NMR analysis showed that the polymer had the required molecular weight (Mn 6500, Mw/Mn 1.08) as expected with two-hydroxyl termini on the chain ends. After drying the hydroxyl terminated poly(D/L-lactide) under vacuum and azeotrope distillation over toluene (to ensure the moisture free prepolymer), 8 g of succinic anhydride (sublimed under vacuum) was mixed with the polymer and the mixture was heated to 130° C. for 8 hours.

The polymer was dissolved in cold dichloromethane. 0.500 ml of water was introduced and the solution was stirred for 1 hour. The water was separated by a globe-shaped separatory funnel. The washing of the polymer solution was carried out three times to remove the unreacted succinic anhydride (by identifying the disappearance of the anhydride peak (1820 cm⁻¹) on the FTIR spectrum). The polymer was recovered from precipitation of the polymer in cold diethyl ether. The yield of the polymer was 105 g.

### EXAMPLE 2

### Synthesis of an α-ω,-di anhydride polylactide prepolymer and its polymerization to yield a polvanhydride based on the dolylactide

The steps in this example are described with reference to FIG. 2. The α-ω,-dicarboxylic acid polylactide (D/L-lactide) from example 1 (105 g) was heated with 600 ml acetic anhydride (chemical purity +99%) to 100° C. for 8 hours, then the mixture was applied vacuum to remove the excess of acetic anhydride. Once all the unreacted acetic anhydride was removed under vacuum, the obtained polymer was added to 10 mg of calcium oxide, and under Argon protection, the mixture was cooked at 165° C. until melting. The temperature was increased to 180° C. for 4 hours with vacuum removal of acetic anhydride. Finally, the temperature was maintained at 180° C. for 8 hours to extend the molecular weight to a maximum. The resulting polyanhydride was pure enough for our applications. The molecular weight was estimated from viscosity measurements to be about 40,000 to 80,000. The polymer has a molecular weight that is high enough to draw flexible fibers from the polymer.

Additional anhydrides were similarly prepared using (i) the α-ω,-di carboxylic acid of a poly lactide pre-polymer having molecular weight 500; (ii) the α-ω,-di carboxylic acid of a poly lactide prepolymer having molecular weight of 1000.

### EXAMPLE 3

### Synthesis of a poly(ethylene glycol) based polyanhydride

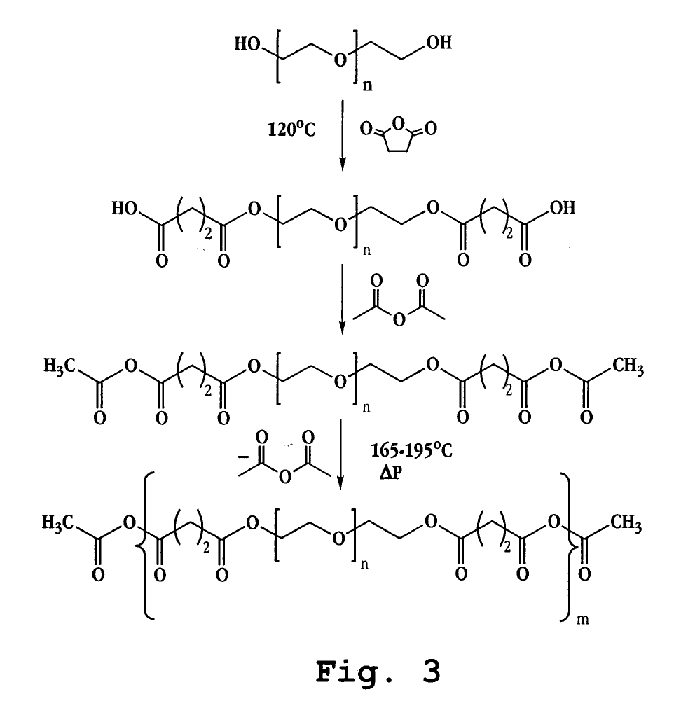

The steps in this example are described with reference to FIG. 3.120 g of poly(ethylene glycol), Sample lot # P4790-EG2OH with Mn=3400, was dissolved in 300 ml dry toluene at 45° C. Azeotropic distillation of toluene was applied to remove the moisture in the sample. After almost all of the toluene was removed, 14 g of succinic anhydride was added, and the mixture was heated to 120° C. under argon protection. The reaction was completed after 4 hours at this temperature. The polymer was dissolved in cold dichloromethane. 500 ml of water was introduced, and the solution was stirred for 1 hour. The water was separated out using a globe-shaped separatory funnel. The washing of the polymer solution was carried out three times to remove the unreacted succinic anhydride (identified by the disappearance of the anhydride peak (1820 cm⁻¹) on the FTIR spectrum). The polymer was recovered by precipitating the polymer in cold diethyl ether. The yield of the polymer was 110 g.

Dry α-ω,- dicarboxy terminated PEG was mixed with acetic anhydride (chemical purity +99%), and the solution was refluxed for 8 hours. Then, the vacuum was applied to remove the excess acetic anhydride. The highly viscous mass material was added with 50 mg of calcium oxide, and under argon protection, the mixture was cooked at 165° C. until melting. The temperature was maintained at 180° C. for 4 hours with vacuum removal of acetic anhydride. Finally, the temperature was maintained at 195° C. for 8 hours to extend the molecular weight to a maximum. The resulting polyanhydride was pure enough for our applications. The molecular weight was estimated from viscosity measurements to be about 30,000 to 50,000.

### EXAMPLE 4

Synthesis of a polyanhydride copolymer containing polylactide and 1.3-bis(carboxyphenoyxy)propane block component (or, drug-containing polymer component)

The steps in this example are described with reference to FIGS. 4 and 5, and can be used in the same or similar manner for drugs such as, for example, salicylates and cyclooxygenase (COX) inhibitors such as, for example, those taught in U.S. Pat. Nos. 6,468,519 and 6,685,928, each of which is hereby incorporated herein by reference in its entirety.

A known quantity of a bis(carboxyphenoxy)alkane dianhydride which, in this case was 1,3-bis(carboxyphenoxy)propane dianhydride, was mixed with a polylactide dianhydride prepolymer by weight, and the mixture was heated under argon in the presence of a CaO catalyst. The polymerization temperature was kept at 150° C. for 2 h under a continuous argon atmosphere to remove the liberated acetic anhydride side-product.

Finally, vacuum was applied to the mixture, and the temperature was maintained at 180° C. for 2 h. and the The temperature was then maintained at 190° C. for 3 h. The polymerization was stopped by cooling down. The product was isolated in the form of light brown color chunk pieces.

### EXAMPLE 5

### Synthesis of a drug-containing polymer component having a linker that forms amide bonds in the drug-containing polymer

Amoxicillin has an amine functionality that can be used to form a dimer of amoxicillin using a linker, after protecting groups have been added to the carbolxyl and hydroxyl functionalities of the amoxicillin. An acyl halide can be used as the linker to form amide linkages, which are less labile than the ester linkages that were formed in Example 4.

Other drugs that can also be protected to form amide bonds with an acyl halide linker include, but are not limited to, cephalexin, carbidopa, levodopa, and amtenac. Examples of such syntheses are known in the art and can be found, for example, in U.S. Pat. No. 6,486,214, which is hereby incorporated herein by reference in its entirety.

Although the invention has been described with respect to certain methods and applications, it will be appreciated that a variety of changes and modification may be made without departing from the invention as claimed.

It is claimed:

1. A biodegradable polymer composition comprising:
(a) a drug-containing polymer component having a backbone containing one or more therapeutic drugs that are linked together by anhydride linkages, such that molecules of the drug are released upon biodegradation of the component, and
(b) a polyester-based polymer component having a molecular weight ranging from about 2 to about 20 KDaltons;
where the composition is characterized by at least one of (i) a Young's modulus of no greater than about 3 GPa, and (ii) a weight ratio of the polyester-based polymer component between about 80% to about 98%.

2. The composition of claim 1, which comprises a blend of the drug-containing polymer component and the polyester-based polymer component.

3. The composition of claim 1, which has a Young's modulus of between 1.5 and 3 GP.

4. The composition of claim 1, wherein the polyester-based polymer component has an average molecular weight ranging from about 2 to about 10 KDaltons.

5. The composition of claim 4, wherein the polyester-based polymer component comprises a component selected from a group consisting of a poly(lactide), a poly(glycolide), or a poly(caprolactone).

6. The composition of claim 1, wherein the number of anhydride linkages in the drug-containing polymer components is between 2 and 20.

7. The composition of 1, wherein the one or more therapeutic drugs in the drug-containing polymer component comprises a drug selected from a group consisting of salicylic acid, derivatives of salicylic acid, salsalate, diflunisal, ibuprofen, derivatives of ibuprofen, naproxen, ketoprofen, diclofenac, indomethacin, mefenamic acid, ketorolac, and iodinated salicylates.

8. The composition of claim 1, which is formed by linking the drug-containing polymer component and the polyester-based polymer component to one another through anhydride linkages.

9. The composition of claim 8, which further includes a linker unit linking the drug-containing and polyester-based polymer components through anhydride linkages.

10. The composition of claim 1, wherein the linker unit is selected from the group consisting of an oligomer of an ether, an amide, an ester, an anhydride, a urethane, a carbamate, a carbonate, a hydroxyalkanoate, or an azo compound.

11. The composition of claim 10, wherein the linker unit contains in its polymer backbone a second therapeutic drug linked in the backbone by biodegradable linkages.

12. The composition of claim 1, characterized by:
(i) a Young's modulus ranging from about 1.5 to about 3 GPa; and
(ii) a rate of surface degradation that is effective to fully erode a bar of the polymer having dimensions of about 50 microns×50 microns×2 mm, when incubated in phosphate buffered saline at 37° C., within a period ranging from about 5 to about 365 days.

13. The composition of claim 1, wherein the polyester-based polymer component has the form, 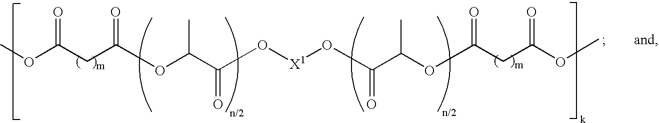
the drug-containing polymer component has the form, 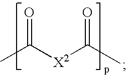
where,
X¹ comprises a component selected from a group consisting of a substituted, unsubstituted, hetero-, straight-chained, branched, cyclic, saturated or unsaturated aliphatic radical; a substituted, unsubstituted, or hetero-aromatic radical; a releasable agent; or a combination thereof; such that X¹ has a molecular weight of less than about 2,000 Daltons;
X² comprises a therapeutic agent and has a molecular weight of less than about 10 KDaltons, wherein the component of X¹ is the same as, or different than, the releasable agent of X²;
k and p are integers selected such that the composition comprised of about 80% to about 98% by weight of the polyester-based polymer component;
m is an integer ranging from 1 to 10; and
n is an integer selected such that the molecular weight of each polyester-based polymer component ranges from about 2 to about 20 KDaltons.

14. The composition of claim 1, wherein the polyester-based polymer component has the form, 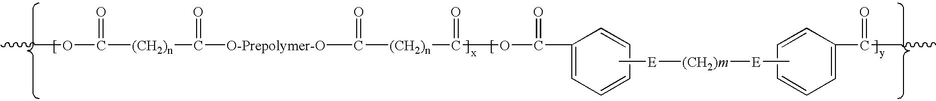
wherein, 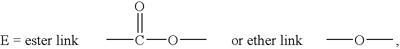
where E is a para ester or a meta or para ether linkage, and the pre-polymer is an α-ω,-dihydroxy terminated polyester or polyether polymer having a molecular weight in a selected range between 1 to 10 Kdaltons;
x=80% to 98% by weight, y=20% to 2% by weight, n=2 to 4, m=2 to 10; and the average total number of anhydride linkages is a selected number in the range between 5-30.

15. The composition of claim 14, wherein the linked phenoxy structure in the polymer is 1,3-bis(p-carboxyphenoxy)propane anhydride, and is present in the polymer in at least 2% by weight.

16. The composition of claim 14, wherein the pre-polymer has an average molecular weight greater than 5 Kdaltons and less than 10 Kdaltons, and an average total number of anhydride linkages between 8 and 12.

17. The polymer of claim 14, wherein the pre-polymer is an α-ω,-dihydroxy terminated polylactide, poly(ε-caprolactone) or polyglycolide polymer.

18. A biodegradable polymer composition comprising:
a drug-containing polymer component having a backbone containing one or more therapeutic drugs that are linked together by biodegradable linkages, such that molecules of the drug are released upon biodegradation of the composition, and having the form, 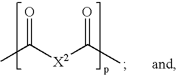
a polyester-based polymer component having an average molecular weight of between 2 and about 20 KDaltons and having the form, 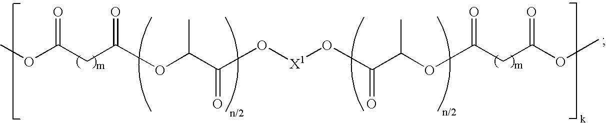
where,
polymer components are linked together through anhydride linkages to form a polyanhydride composition,
X¹ comprises a component selected from a group consisting of a substituted, unsubstituted, hetero-, straight-chained, branched, cyclic, saturated or unsaturated aliphatic radical; a substituted, unsubstituted, or hetero- aromatic radical; a releasable agent; or a combination thereof; such that X¹ has a molecular weight of less than about 2,000 Daltons;
X² comprises a therapeutic agent and has a molecular weight of less than about 10 KDaltons, wherein the component of X¹ is the same as, or different than, the releasable agent of X²;
k and p are integers selected such that the poly(anhydride) is comprised of about 80% to about 98% by weight of the polyester-based polymer component;
m is an integer ranging from 1 to about 10; and,
n is an integer selected such that the molecular weight of each polyester-based polymer component ranges from about 2 to about 20 KDaltons.

19. The composition of claim 18, which comprises a blend of the drug-containing polymer component and the polyester-based polymer component.

20. The composition of claim 18, wherein the polyester-based polymer component has an average molecular weight ranging from about 2 to about 10 KDaltons.

21. The composition of claim 18, wherein the polyester-based polymer component has an average molecular weight ranging from about 5 to about 7 KDaltons.

22. The composition of claim 18, wherein the polyester-based polymer component comprises a component selected from a group consisting of a poly(lactide), a poly(glycolide), or a poly(caprolactone).

23. The composition of claim 18, which is characterized by:
(i) a Young's modulus ranging from about 1.5 to about 3 GPa; and
(ii) a rate of surface degradation that is effective to fully erode a bar of the polymer having dimensions of about 50 microns×50 microns×2 mm, when incubated in phosphate buffered saline at 37° C., within a period ranging from about 5 to about 365 days.

24. The composition of claim 18, which is formed by linking the drug-containing component and the polyester-based component to one another through anhydride linkages.

25. The composition of claim 24, which further includes a linker unit linking the drug-containing and polyester-based polymer components through anhydride linkages.

26. The composition of claim 18, wherein the polyester-based polymer component has the form, 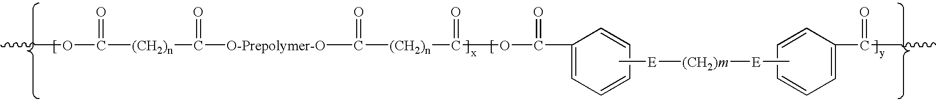
wherein, 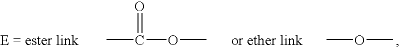
where E is a para ester or a meta or para ether linkage, and the pre-polymer is an α-ω,-dihydroxy terminated polyester or polyether polymer having a molecular weight in a selected range between 1 to 10 Kdaltons;
x=80% to 98% by weight, y=20% to 2% by weight, n=2 to 4, m=2 to 10; and the average total number of anhydride linkages is a selected number in the range between 5-30.

27. The composition of claim 18, wherein the linked phenoxy structure in the polymer is 1,3-bis(p-carboxyphenoxy)propane anhydride, and is present in the polymer in at least 2% by weight.

28. The composition of claim 18, wherein the pre-polymer has an average molecular weight greater than 5 Kdaltons and less than 10 Kdaltons, and an average total number of anhydride linkages between 8 and 12.

29. The polymer of claim 18, wherein the pre-polymer is an α-ω,-dihydroxy terminated polylactide, poly(ε-caprolactone) or polyglycolide polymer.

30. A method of producing a biodegradable polymer composition containing a releasable therapeutic drug and having a selected Young's modulus of no greater than about 3.5 GPa, comprising:
mixing a drug-containing polymer component having a backbone containing terminal anhydride groups and one or more therapeutic drugs that are linked together by biodegradable linkages, such that molecules of the drug are released upon biodegradation of the composition, with a polyester-based polymer component having a molecular weight ranging from about 2 to about 10 KDaltons and terminal anhydride groups to produce a mixture, where the weight percentage of the polyester-based polymer component ranges from about 80% to about 98% of the mixture.

31. The method of claim 30, which further includes polymerizing the polymer components under time and temperature conditions effective to produce a polyester-based polyanhydride having a selected average number of anhydride linkages in the range of about 5 to about 25.

32. The method of claim 30, wherein the polyester-based polymer components have an average molecular weight ranging from about 5 to about 7 KDaltons.

33. The method of claim 30, wherein the polyester-based polymer component comprises a component selected from a group consisting of a poly(lactide), a poly(glycolide), or a poly(caprolactone).

34. The method of claim 30, wherein the polyester-based polymer component has the form, 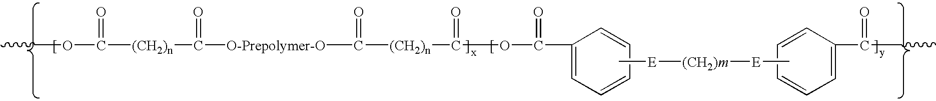
wherein, 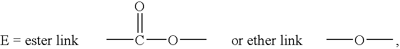
where E is a para ester or a meta or para ether linkage, and the pre-polymer is an α-ω,-dihydroxy terminated polyester or polyether polymer having a molecular weight in a selected range between 1 to 10 Kdaltons;
x=80% to 98% by weight, y=20% to 2% by weight, n=2 to 4, m=2 to 10; and the average total number of anhydride linkages is a selected number in the range between 5-30.

35. The method of claim 34, wherein the linked phenoxy structure is 1,3-bis(p-carboxyphenoxy)propane anhydride, and is present in the polymer in at least 2% by weight.

36. The method of claim 34, wherein the pre-polymer has an average molecular weight greater than 5 Kdaltons and less than 10 Kdaltons, and an average total number of anhydride linkages between 8 and 12.

37. The method of claim 34, wherein the pre-polymer is an α-ω,-dihydroxy terminated polylactide, polyε-caprolactone or polyglycolide polymer.

38. An expandable intravascular stent comprising an expandable stent body and an outer, drug-eluting coating comprising:
(a) the biodegradable polymer composition of claim 1; and
(b) an anti-restenosis compound.

39. The stent of claim 38, wherein the stent's expandable body is formed of a poly(ester)-based polymer component linked by anhydride linkages, and having a Young's modulus of less than about 3 GPa.

40. The stent of claim 38, wherein the biodegradable polymer composition of claim 1 comprises the biodegradable polymer composition of claim 14.

41. A stent comprising an expandable body; a first layer on a surface of the stent comprising rapamycin, or a derivative thereof; and a second layer on at least a portion of the first layer comprising an NSAID; wherein, at least one of the expandable body, the first layer, or the second layer comprises a biodegradable polymer composition comprising:
(a) a drug-containing polymer component having a backbone containing one or more therapeutic drugs that are linked together by biodegradable linkages, such that molecules of the drug are released upon biodegradation of the composition; and,
(b) a polyester-based polymer component having a molecular weight ranging from about 2 to about 20 KDaltons;
where, the composition is characterized by at least one of (i) a Young's modulus of no greater than about 3 GPa, (ii) a rate of surface degradation that is effective to fully erode a bar of the polymer having dimensions of about 50 microns×50 microns×2 mm, when incubated in phosphate buffered saline at 37° C., within a period of about 5 to about 365 days, or (iii) a weight ratio of the polyester-based polymer component that is between about 80% to about 98%.

42. The stent of claim 41, wherein the biodegradable polymer composition comprises the biodegradable polymer composition of claim 14.
